(19) United States
(12) Patent Application Publication
(10) Pub. No.: US 2024/0207113 A1
(43) Pub. Date: Jun. 27, 2024

(54) Wound Dressing Supply Holding Device And Method Of Use

(71) Applicant: Jeff Rubel, Brooklyn, OH (US)

(72) Inventor: Jeff Rubel, Brooklyn, OH (US)

(21) Appl. No.: 18/087,340

(22) Filed: Dec. 22, 2022

(51) Int. Cl. A61F 15/00

(52) U.S. Cl. CPC A61F 15/002

(57) ABSTRACT

A wound dressing supply holding device enabling as needed access to wound dressing supplies includes a shell, which has an open top and which is sized and shaped complementarily to a stack of the bandages, which can be inserted into the shell through the open top. A respective bandage can be removed, as needed, from the stack of the bandages. A clip that is attached to a rear face of the shell selectively attaches the shell to a waistband or belt of a user. A hollow disc is attached by an inner wall to a front face of the shell. An opening is positioned in an upper sidewall of the hollow disc and allows for insertion of a roll of surgical tape into the hollow disc so that the surgical tape is available to the user to affix the respective bandage to cover a wound.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to wound dressing kits and more particularly pertains to a new wound dressing kit enabling as needed access to wound dressing supplies. The present invention discloses a shell for holding bandages that can be clipped to a belt and to which is attached a hollow disc that can hold a roll of surgical tape.

### (2) Description of Related Art Including Information Disclosed Under 37 CFR 1.97 and 1.98

The prior art relates to wound dressing kits, which may comprise wound dressing kits configured as sleeves, belt attachable spool holding devices, bandage dispensers, and the like. What is lacking in the prior art is a shell for holding bandages that can be clipped to a belt and to which is attached a hollow disc that can hold a roll of surgical tape.

### BRIEF SUMMARY OF THE INVENTION

An embodiment of the disclosure meets the needs presented above by generally comprising a shell, which has an open top and which is sized and shaped complementarily to a stack of bandages. The open top is configured for insertion of the stack of the bandages into the shell and for selective extraction of a respective bandage from the stack of the bandages. A clip is attached to a rear face of the shell and is configured to attach the shell to a waistband or belt of a user so that the shell is removably attached to the user. A hollow disc is attached by an inner wall to a front face of the shell. An opening is positioned in an upper sidewall of the hollow disc and is configured for insertion of a roll of surgical tape into the hollow disc so that the surgical tape is available to the user to affix the respective bandage to cover a wound.

Another embodiment of the disclosure includes a wound dressing supply holding system, which comprises a wound dressing supply holding device, according to the disclosure above, having a stack of bandages and a roll of surgical tape positioned in a shell and a hollow disc of the wound dressing supply holding device.

Yet another embodiment of the disclosure includes a method of dressing a wound comprising first and second provision steps, which entails providing a stack of bandages and a roll of surgical tape and providing a wound dressing supply holding device, according to the disclosure above. Preparatory steps of the method are inserting the roll of the surgical tape into the hollow disc and inserting the stack of the bandages into the shell, respectively. The wound dressing supply holding device then is attached to a belt using the clip. Operational steps of the method are extracting a bandage from the shell, removing a covering from the bandage, positioning the bandage over a wound, extending surgical tape from the roll of the surgical tape, severing the surgical tape, and applying the surgical tape to the bandage and adjacent skin to affix the bandage over the wound.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front isometric perspective view of a wound dressing supply holding device according to an embodiment of the disclosure.

FIG. 2 is a rear isometric perspective view of an embodiment of the disclosure.

FIG. 3 is a top view of an embodiment of the disclosure.

FIG. 4 is a front view of an embodiment of the disclosure.

FIG. 5 is a cross-sectional view of an embodiment of the disclosure.

FIG. 6 is an in-use view of an embodiment of the disclosure.

FIG. 7 is a flow diagram for a method utilizing an embodiment of the disclosure.

### DETAILED DESCRIPTION OF THE INVENTION

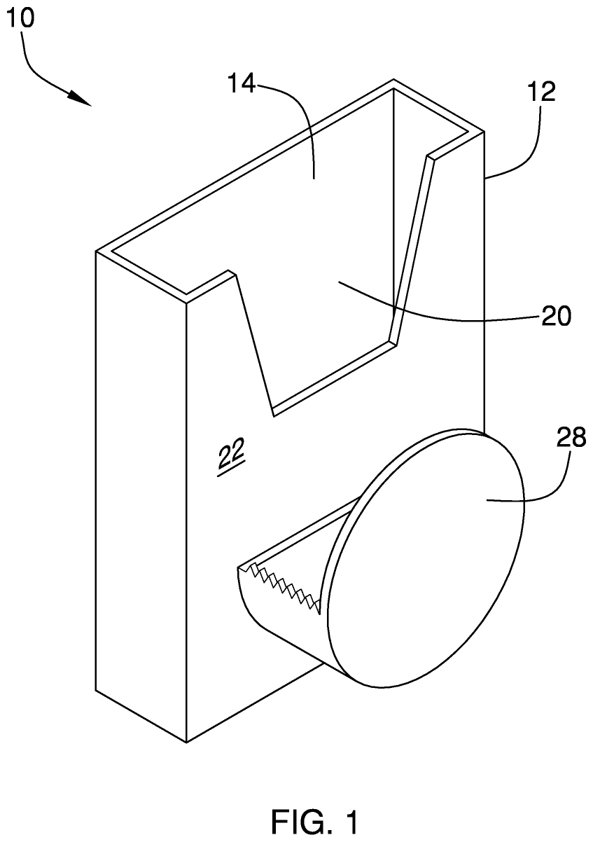

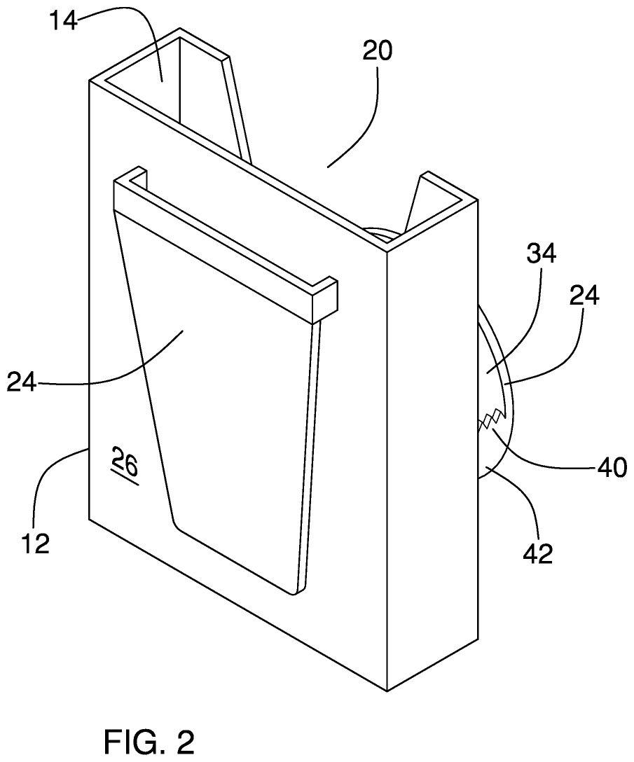

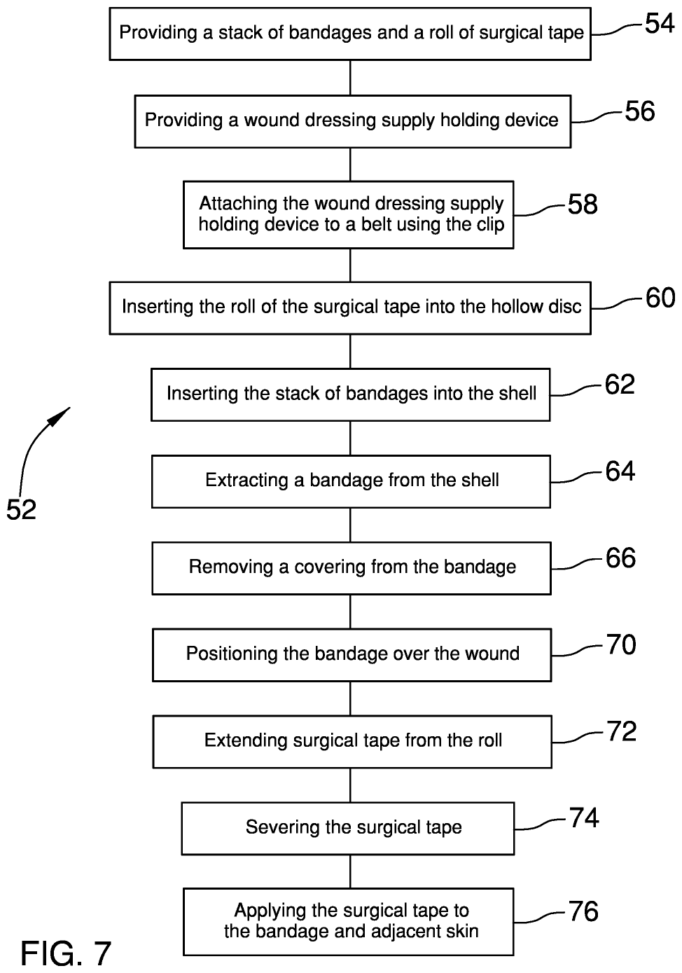

With reference now to the drawings, and in particular to FIGS. 1 through 7 thereof, a new wound dressing kit embodying the principles and concepts of an embodiment of the disclosure and generally designated by the reference numeral 10 will be described.

As best illustrated in FIGS. 1 through 7, the wound dressing supply holding device 10 generally comprises a shell 12, which has an open top 14 and which is sized and shaped complementarily to a stack 16 of bandages 18. The open top 14 is configured for insertion of the stack 16 of the bandages 18 into the shell 12 and for selective extraction of a respective bandage 18 from the stack 16 of the bandages 18. The shell 12 is substantially cuboid, as shown in FIGS. 1-4. A cutout 20 extends from the open top 14 into a front face 22 of the shell 12 and is configured for insertion of one or more digits of a hand of a user to facilitate extraction of the respective bandage 18 from the stack 16 of the bandages 18. As shown in FIG. 4, the cutout 20 is trapezoidal, although the present invention also anticipates the cutout 20 being rectangular, semicircular, and the like.

The present invention also anticipates a set of walls (not shown), which is attached to and positioned in the shell 12 to define a plurality of compartments. Each compartment could be sized for insertion of bandages 18 of a respective size, an antimicrobial ointment container, an antihemorrhagic agent container, or the like.

A clip 24 is attached to a rear face 26 of the shell 12 and is configured to attach the shell 12 to a waistband or belt of the user so that the shell 12 is removably attached to the user. Other means for attaching the shell 12 to a waist of the user also are anticipated by the present invention, such as, but not limited to, hook and loop straps that fasten around belts, magnets that fasten to metal plates on belts, and the like.

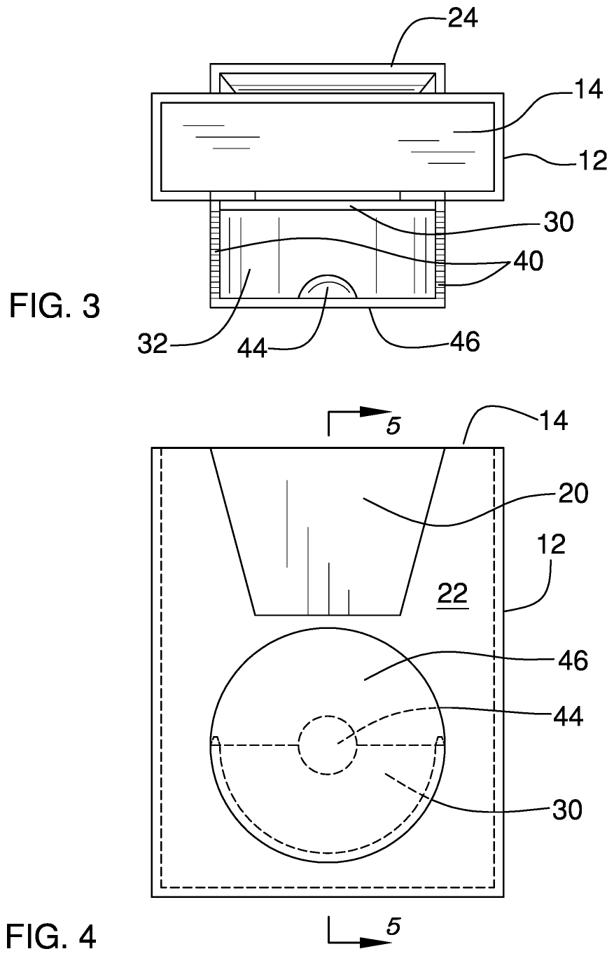

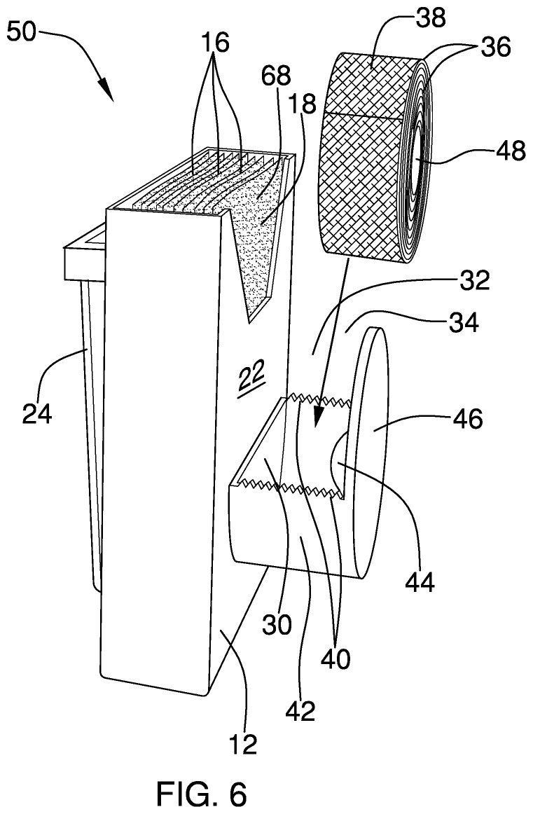

A hollow disc 28 is attached by its inner wall 30 to the front face 22 of the shell 12. As shown in FIG. 3, an opening 32 is positioned in an upper sidewall 34 of the hollow disc 28 and is configured for insertion of a roll 36 of surgical tape 38 into the hollow disc 28 so that the surgical tape 38 is available to the user to affix the respective bandage 18 to cover a wound. At least one opposed end 40 of opposed ends 40 of a lower sidewall 42 of the hollow disc 28 is serrated. The at least one opposed end 40 is configured to selectively sever surgical tape 38 from the roll 36 of the surgical tape 38. As shown in FIGS. 3 and 6, both opposed ends 40 are serrated so that the surgical tape 38 can be severed easily when the wound dressing supply holding device 10 is placed on either of a left hip or a right hip of the user.

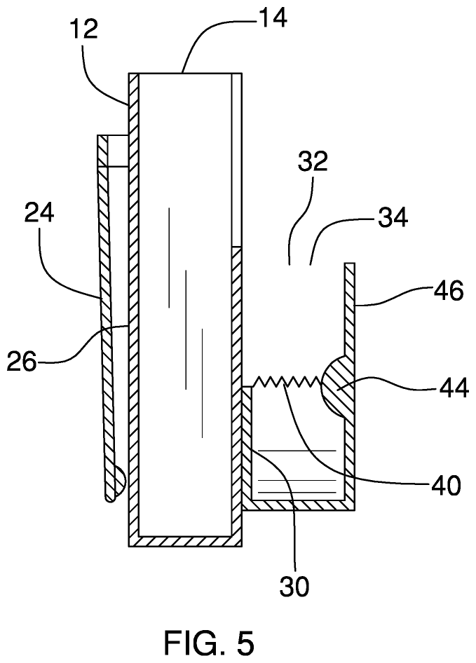

As shown in FIG. 5, a nub 44 is attached to an outer wall 46 of the hollow disc 28 and extends axially into the hollow disc 28. The nub 44 is configured for insertion into a tube 48 of the roll 36 of the surgical tape 38 as the roll 36 of the surgical tape 38 is inserted into the hollow disc 28 so that retention of the roll 36 of the surgical tape 38 within the hollow disc 28 is facilitated. The nub 44 may be substantially hemispherical, thereby facilitating insertion of the roll 36 of the surgical tape 38.

The nub 44, the shell 12, and the hollow disc 28 comprise plastic and one or more of silver, zinc, a natural microbial agent, and a synthetic microbial agent. A wide range of natural and nature inspired antimicrobial agents can be incorporated into the nub 44, the shell 12, and the hollow disc 28, such as, but not limited to, silanes, polymeric biocides, and the like. The nub 44, the shell 12, and the hollow disc 28 thus are rendered antimicrobial. The wound dressing supply holding device 10 stores the stack 16 of the bandages 18 and the roll 36 of the surgical tape 38 so they are maintained clean and readily accessible for use in covering wounds, thereby reducing a risk of infection and improving clinical outcomes. The wound dressing supply holding device 10 facilitates bandaging by a user using only one hand. Often, one hand of the user is required for compression on a wound to stem bleeding, leaving only one hand available for manipulating a bandage 18 and the surgical tape 38.

The present invention also anticipates a wound dressing supply holding system 50 comprising a wound dressing supply holding device 10, according to the specification above, having a stack 16 of bandages 18 and a roll 36 of surgical tape 38 positioned in a shell 12 and a hollow disc 28 of the wound dressing supply holding device 10.

In use, the wound dressing supply holding device 10 enables a method of dressing a wound 52. The method 52 comprises a first provision step 54, which entails providing a stack 16 of bandages 18 and a roll 36 of surgical tape 38. A second provision step 56 of the method 52 is providing a wound dressing supply holding device 10, according to the specification above. A donning step 58 of the method 52 is attaching the wound dressing supply holding device 10 to a belt using the clip 24. A first preparation step 60 and a second preparation step 62 of the method 52 are inserting the roll 36 of the surgical tape 38 into the hollow disc 28 and inserting the stack 16 of the bandages 18 into the shell 12, respectively.

A first operational step 64 and a second operational step 66 of the method 52 are extracting a respective bandage 18 from the shell 12 and removing a covering 68 from the respective bandage 18. A third operational step 70 of the method 52 is positioning the respective bandage 18 over a wound. A fourth operational step 72 of the method 52 and a fifth operational step 74 are extending surgical tape 38 from the roll 36 of the surgical tape 38 and severing the surgical tape 38 by pulling it against the at least one opposed end 40 of the lower sidewall 42. A sixth operational step 76 of the method 52 is applying the surgical tape 38 to the respective bandage 18 and adjacent skin to affix the respective bandage 18 over the wound.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A wound dressing supply holding device comprising:
a shell having an open top, the shell being sized and shaped complementarily to a stack of bandages, wherein the open top is configured for insertion of the stack of the bandages into the shell and for selective extraction of a respective bandage from the stack of the bandages;
a clip attached to a rear face of the shell, wherein the clip is configured for attaching the shell to a waistband or belt of a user, such that the shell is removably attached to the user;
a hollow disc attached by an inner wall to a front face of the shell; and
an opening positioned in an upper sidewall of the hollow disc, wherein the opening is configured for insertion of a roll of surgical tape into the hollow disc, such that the surgical tape is available to the user for affixing the respective bandage to cover a wound.

2. The wound dressing supply holding device of claim 1, wherein the shell is substantially cuboid.

3. The wound dressing supply holding device of claim 1, further including a cutout extending from the open top into the front face of the shell, wherein the cutout is configured for insertion of one or more digits of a hand of the user for facilitating extraction of the respective bandage from the stack of the bandages.

4. The wound dressing supply holding device of claim 1, further including at least one opposed end of opposed ends of a lower sidewall of the hollow disc being serrated, wherein the at least one opposed end is configured for selectively severing surgical tape from the roll of the surgical tape.

5. The wound dressing supply holding device of claim 1, further including a nub attached to an outer wall of the hollow disc and extending axially into the hollow disc, wherein the nub is configured for insertion into a tube of the roll of the surgical tape as the roll of the surgical tape is inserted into the hollow disc, such that retention of the roll of the surgical tape within the hollow disc is facilitated.

6. The wound dressing supply holding device of claim 5, wherein the nub is substantially hemispherical.

7. The wound dressing supply holding device of claim 5, wherein the nub, the shell, and the hollow disc comprise plastic and one or more of silver, zinc, a natural microbial agent, and a synthetic microbial agent, such that the nub, the shell, and the hollow disc are antimicrobial.

8. The wound dressing supply holding device of claim 1, further including:
the shell being substantially cuboid;
a cutout extending from the open top into the front face of the shell, wherein the cutout is configured for insertion of one or more digits of a hand of the user for facilitating extraction of the respective bandage from the stack of the bandages;
at least one opposed end of opposed ends of a lower sidewall of the hollow disc being serrated, wherein the at least one opposed end is configured for selectively severing surgical tape from the roll of the surgical tape;
a nub attached to an outer wall of the hollow disc and extending axially into the hollow disc, wherein the nub is configured for insertion into a tube of the roll of the surgical tape as the roll of the surgical tape is inserted into the hollow disc, such that retention of the roll of the surgical tape within the hollow disc is facilitated, the nub being substantially hemispherical; and
the nub, the shell, and the hollow disc comprising plastic and one or more of silver, zinc, a natural microbial agent, and a synthetic microbial agent, such that the nub, the shell, and the hollow disc are antimicrobial.

9. A wound dressing supply holding system comprising:
a wound dressing supply holding device comprising:
a shell having an open top, the shell being sized and shaped complementarily to a stack of bandages, wherein the open top is configured for insertion of the stack of the bandages into the shell and for selective extraction of a respective bandage from the stack of the bandages,
a clip attached to a rear face of the shell, wherein the clip is configured for attaching the shell to a waistband or belt of a user, such that the shell is removably attached to the user,
a hollow disc attached by an inner wall to a front face of the shell, and
an opening positioned in an upper sidewall of the hollow disc, wherein the opening is configured for insertion of a roll of surgical tape into the hollow disc, such that the surgical tape is available to the user for affixing the respective bandage to cover a wound;
a stack of bandages positioned in the shell; and
a roll of surgical tape positioned in the hollow disc.

10. A method of dressing a wound comprising the steps of:
providing a stack of bandages and a roll of surgical tape;
providing a wound dressing supply holding device comprising:
a shell having an open top, the shell being sized and shaped complementarily to the stack of the bandages,
a clip attached to a rear face of the shell, wherein the clip is configured for attaching the shell to a waistband or belt of a user, such that the shell is removably attached to the user,
a hollow disc attached by an inner wall to a front face of the shell, and
an opening positioned in an upper sidewall of the hollow disc, at least one opposed end of opposed ends of a lower sidewall of the hollow disc being serrated;
inserting the roll of the surgical tape into the hollow disc;
inserting the stack of the bandages into the shell;
attaching the wound dressing supply holding device to a belt using the clip;
extracting a respective bandage from the shell;
removing a covering from the respective bandage;
positioning the respective bandage over a wound;
extending surgical tape from the roll of the surgical tape;
severing the surgical tape by pulling it against the at least one opposed end of the lower sidewall; and
applying the surgical tape to the respective bandage and adjacent skin to affix the respective bandage over the wound.
